(19) United States
(12) Patent Application Publication
(10) Pub. No.: US 2014/0318277 A1
(43) Pub. Date: Oct. 30, 2014

(54) SAMPLING DEVICE ADAPTED FOR SAMPLING AIRBORNE COMPONENTS

(71) Applicant: ATLAS TECHNOLOGY CORP., New Taipei City (TW)

(72) Inventors: Hung KANG, New Taipei City (TW); David LIU, New Taipei City (TW)

(73) Assignee: ATLAS TECHNOLOGY CORP., New Taipei City (TW)

(21) Appl. No.: 14/069,783

(22) Filed: Nov. 1, 2013

(30) Foreign Application Priority Data
Apr. 26, 2013 (TW) 102207777

(51) Int. Cl. G01N 1/22; G01N 33/00

(52) U.S. Cl. CPC G01N 1/2202; G01N 33/0004
USPC 73/863.21

(57) ABSTRACT

A sampling device includes a gas guiding unit having upper and lower guiding grooves and a liquid guiding unit abutting against the gas guiding unit and having a passage groove that communicates fluidly with the upper and lower guiding grooves. The liquid guiding unit includes a liquid-retaining mechanism including a liquid-retaining net that enables liquid to form a liquid membrane through capillary action. Gas introduced into the passage groove passes through the liquid membrane where airborne components contained therein are dissolved. The liquid containing the dissolved airborne components is extracted from the sampling device as a test sample.

### CROSS-REFERENCE TO RELATED APPLICATION

This application claims priority of Taiwanese Application No. 102207777, filed on Apr. 26, 2013.

### BACKGROUND OF THE INVENTION

1. Field of the Invention

The invention relates to a sampling device, particularly to a sampling device adapted for sampling dissolvable airborne components.

2. Description of the Related Art

Airborne Molecular Contamination (AMC) is not only potentially harmful to human bodies, but also impacts negatively on yield rates in fine precision industries such as in semiconductor manufacturing. Due to miniaturization of semiconductor devices, the controlling of AMC is becoming ever more critical.

Currently, AMC detection involves sampling airborne components by having the same dissolved in liquid and analyzing the ion density of the liquid having airborne components dissolved therein using ion chromatography. For easier sampling, Taiwanese Patent No. M378380 discloses a conventional sampling device which uses a dialysis membrane to separate a gas channel from a liquid channel to prevent oily organic gas from attaching to a liquid channel wall that defines the liquid channel, which would otherwise hinder airborne components in the gas channel from being dissolved in liquid in the liquid channel. However, since airborne components have to penetrate the dialysis membrane to reach the liquid, some may be attached to the membrane without ever reaching the liquid, hence affecting the test result.

Another conventional technique utilizes a porous glass coated with TiO₂ nanoparticles to form a super-hydrophilic surface. This conventional technique uses relatively expensive materials and has relatively great manufacturing complexity.

### SUMMARY OF THE INVENTION

Therefore, the object of the present invention is to provide a sampling device adapted for sampling airborne components and capable of eliminating the aforesaid drawbacks of the prior art.

According to the present invention, there is provided a sampling device adapted for sampling airborne components and including a gas guiding unit and a liquid guiding unit.

The gas guiding unit includes a first plate that has a top surface formed with a gas outlet, a bottom surface formed with a gas inlet, and a first abutment surface interconnecting the top and bottom surfaces, and formed with upper and lower guiding grooves. The upper and lower guiding grooves are proximate respectively to the top and bottom surfaces, and communicate fluidly and respectively the gas outlet and the gas inlet.

The liquid guiding unit includes a second plate that has a second abutment surface and a side surface. The second abutment surface abuts against the first abutment surface and is formed with a passage groove that is defined by a groove-defining surface. The passage groove has a top groove portion, a bottom groove portion and an intermediate groove portion that interconnects the top and bottom groove portions, and that has a depth smaller than those of the top and bottom groove portions. The passage groove communicates fluidly the upper and lower guiding grooves of the gas guiding unit, and a side surface formed with a liquid inlet that is in fluid communication with the top groove portion, and a liquid outlet that is in fluid communication with the bottom groove portion.

The liquid guiding unit further includes a liquid-retaining mechanism that includes a liquid-retaining net clinging to the groove-defining surface of the liquid guiding unit so that liquid introduced into the top groove portion via the liquid inlet forms a liquid membrane on the liquid-retaining net through capillary action.

Gas introduced into the lower guiding groove via the gas inlet flows into the upper guiding groove through the passage groove with airborne components contained therein dissolving in the liquid membrane. The liquid containing the dissolved airborne components flows into the bottom groove portion and exits the sampling device via the liquid outlet.

### BRIEF DESCRIPTION OF THE DRAWINGS

Other features and advantages of the present invention will become apparent in the following detailed description of the preferred embodiment with reference to the accompanying drawings, of which:

FIG. 1 is a side schematic view of a sampling device according to the preferred embodiment of the present invention;

FIG. 2 is a view of a gas guiding unit of the preferred embodiment, looking from the right hand side to the left hand side of FIG. 1;

FIG. 3 is a top view of the gas guiding unit;

FIG. 4 is a view of a liquid guiding unit of the preferred embodiment, looking from the left hand side to the right hand side of FIG. 1; and

FIG. 5 is a sectional view of the liquid guiding unit taken along line V-V of FIG. 4.

### DETAILED DESCRIPTION OF THE PREFERRED EMBODIMENT

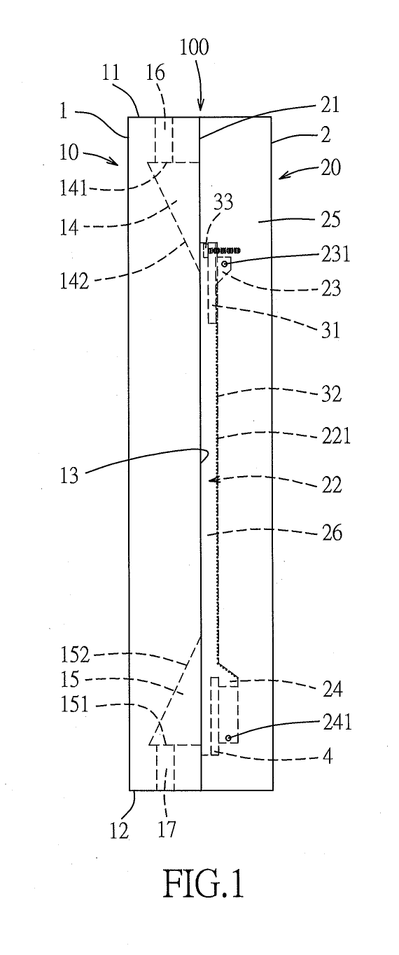

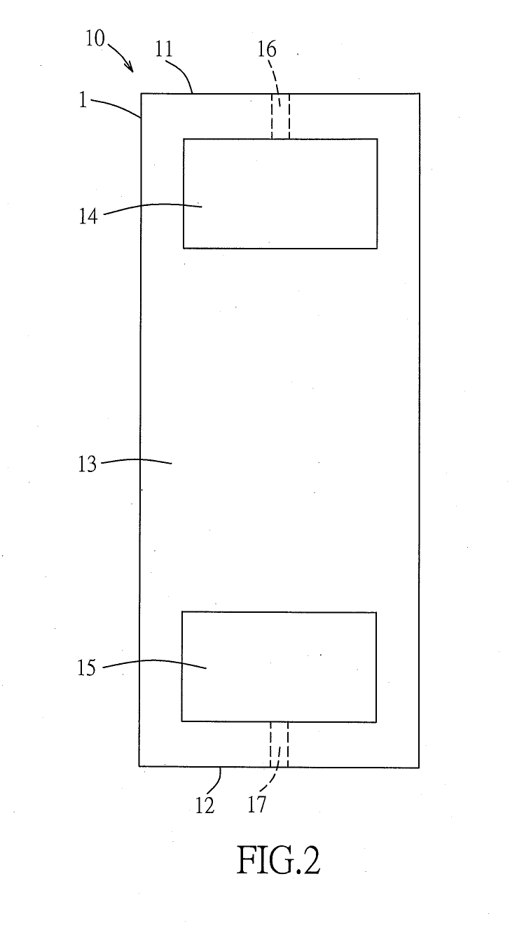

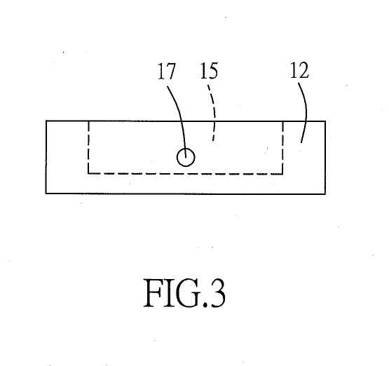

With reference to FIGS. 1 to 3, a sampling device 100 adapted for sampling airborne components according to the present invention includes a gas guiding unit 10 and a liquid guiding unit 20. The gas guiding unit 10 includes a first plate 1 that has a top surface 11, a bottom surface 12, a first abutment surface 13, an upper guiding groove 14, a lower guiding groove 15, a gas outlet 16 and a gas inlet 17. The first abutment surface 13 interconnects the top surface 11 and the bottom surfaces 12. The upper guiding groove 14 and the lower guiding groove 15 are formed in the first abutment surface 13 and proximate respectively to the top surface 11 and the bottom surface 12, and communicate respectively and fluidly the gas outlet 16 and the gas inlet 17. The upper guiding groove 14 is defined by a horizontal surface 141 and an inclined groove surface 142; the lower guiding groove 15 is defined by a horizontal surface 151 and an inclined groove surface 152. The horizontal groove surfaces 141, 151 are perpendicular to the first abutment surface 13. The inclined groove surfaces 142, 152 extend and incline respectively from the horizontal groove surfaces 141, 151 to the first abutment surface 13. The gas outlet 16 and the gas inlet 17 are respectively formed in the top and bottom surfaces 11, 12, and each extend through the horizontal groove surface 141, 151 defining the respective one of the upper and lower guiding grooves 14, 15. The gas inlet 17 is adapted to be connected to a gas inlet pipe (not shown) to introduce gas to be tested, and the gas outlet 16 is adapted to be connected to a gas outlet pipe (not shown) for the gas introduced into the gas inlet 17 to exit therethrough.

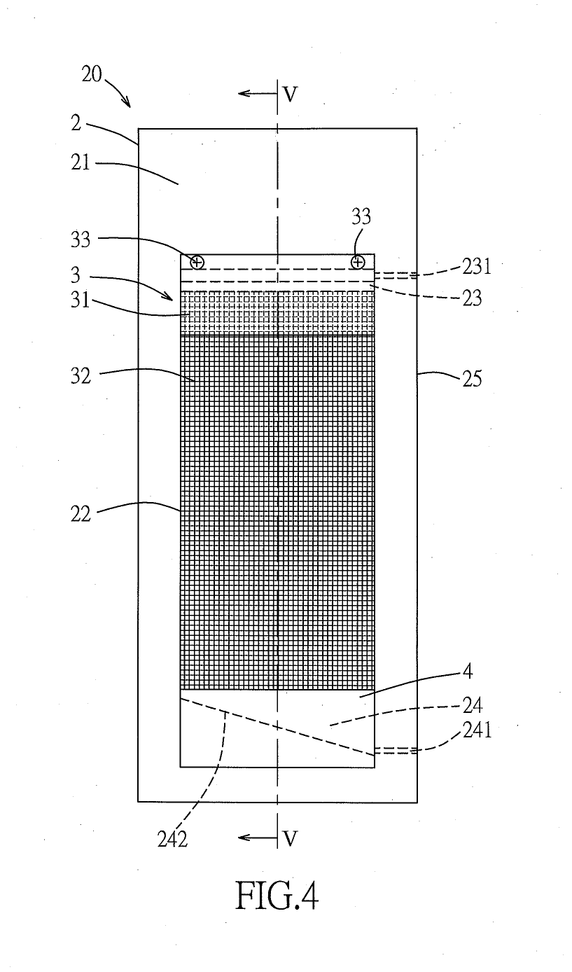

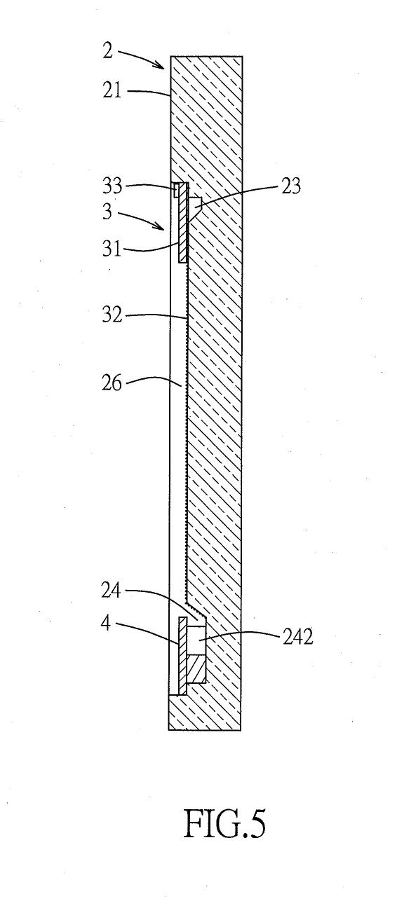

With reference to FIGS. 1, 4 and 5, the liquid guiding unit 20 includes a second plate 2 and a liquid-retaining mechanism 3. The second plate 2 has a second abutment surface 21 abutting against the first abutment surface 13, a side surface 25, and a passage groove 22 defined by a groove-defining surface 221. The passage groove 22 is formed in the second abutment surface 21, and has a top groove portion 23, a bottom groove portion 24 and an intermediate groove portion 26 that interconnects the top and bottom groove portions 23, 24, and that has a depth smaller than those of the top and bottom groove portions 23, 24. The passage groove 22 communicates fluidly the upper and lower guiding grooves 14, 15 of the gas guiding unit 10. The side surface 25 is formed with a liquid inlet 231 that is in fluid communication with the top groove portion 23 and that is adapted to be connected to a liquid inlet pipe (not shown) for introducing liquid into the top groove portion 23. The side surface 25 is further formed with a liquid outlet 241 that is in fluid communication with the bottom groove portion 24. The groove-defining surface 221 has an inclined bottom section 242 bordering a bottom end of the bottom groove portion 24. The inclined bottom section 242 has an end that is proximate to the side surface 25 of the second plate 2 and that is not higher than the liquid outlet 241 and an opposite end that is distal from the side surface 25 of the second plate 2 and that is above the liquid outlet 241. The liquid outlet 241 is adapted to be connected to a liquid outlet pipe (not shown) which can be connected to a liquid pump (not shown).

The liquid guiding unit 20 further includes a stop plate 4 connected watertightly to the second plate 2 and cooperating with the second plate 2 to define the bottom groove portion 24. In this embodiment, the liquid inlet and outlet 231, 241 are formed in the same side surface 25, but the liquid inlet and outlet 231, 241 may be disposed on opposite sides should it be required in practice.

The liquid-retaining mechanism 3 is disposed on the second plate 2 and includes an anchor plate 31 and a liquid-retaining net 32. The anchor plate 31 is mounted to the second plate 2 by two screwing bolts 33, and is disposed adjacent to the top groove portion 23 for holding the liquid-retaining net 32 to cling on the groove-defining surface 221. Liquid introduced into the top groove portion 23 via the liquid inlet 231 forms a liquid membrane on the liquid-retaining net 32 through capillary action. Gas introduced into the lower guiding groove 15 via the gas inlet 17 flows into the upper guiding groove 16 through the passage groove 22 with airborne components contained therein dissolving in the liquid membrane. The liquid containing the dissolved airborne components flows into the bottom groove portion 24, is guided by the inclined bottom section 242 of the groove-defining surface 221 to exit the sampling device 100 via the liquid outlet 241, and then may be extracted by the liquid pump as a sample for subsequent use in ion chromatography. The liquid-retaining net 32 preferably has 100 to 400 meshes per square inch and may adopt commercially available mesh used in household screen windows and doors.

The operation of the sampling device 100 according to the preferred embodiment of the present invention is described in detail as follows.

Liquid used for sampling the airborne components is introduced into the top groove portion 23 via the liquid inlet 231, flows down to the liquid-retaining net 32, spreads over the liquid-retaining net 32 to form a liquid membrane through capillary action, slowly flows down to the bottom of the liquid-retaining net 32 and then the bottom groove portion 24, and eventually exits the passage groove 22 via the liquid outlet 241.

Gas containing airborne components is introduced into the lower guiding groove 15 via the gas inlet 17 and guided by the inclined groove surface 152 into the passage groove 22. The gas is in contact with the liquid membrane as the gas flows upwards along the liquid membrane and the airborne components in the air are dissolved in the liquid membrane. After passing the passage groove 22, the gas continues flowing upwards to the upper guiding groove 14 via the inclined groove surface 142 and eventually exits the gas outlet 16.

As described previously, the liquid would flow downwardly along the liquid-retaining net 32, having the airborne components dissolved therein along the way, and is collected at the bottom groove portion 24 and extracted from the liquid outlet 241 for testing for Airborne Molecular Contamination (AMC).

The liquid membrane formed on the liquid-retaining net 32 increases a contacting surface between the liquid and the gas, and the gas is exposed directly to the liquid to effectively increase the airborne component absorption efficiency of the liquid. Comparing to the conventional sampling device, the liquid-retaining net 32 of the present invention provides the same absorption surface with a reduced amount of liquid, thereby saving on the usage of the liquid. In addition, the liquid retaining net 32 is easily accessible, and can be mounted to the second plate 2 with ease, thereby simplifying the manufacturing process and reducing cost.

The sampling device 100 of the present invention can be adapted for use with testing instruments to function together as a sampling and testing system.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A sampling device adapted for sampling airborne components, comprising:
a gas guiding unit including a first plate that has
a top surface formed with a gas outlet,
a bottom surface formed with a gas inlet, and
a first abutment surface interconnecting said top and bottom surfaces, and formed with upper and lower guiding grooves that are proximate respectively to said top and bottom surfaces, and that communicate fluidly and respectively said gas outlet and said gas inlet; and
a liquid guiding unit including
a second plate that has
a second abutment surface abutting against said first abutment surface and formed with a passage groove that is defined by a groove-defining surface, said passage groove having a top groove portion, a bottom groove portion and an intermediate groove portion that interconnects said top and bottom groove portions, and that has a depth smaller than those of said top and bottom groove portions, said passage groove communicating fluidly said upper and lower guiding grooves of said gas guiding unit, and
a side surface formed with a liquid inlet that is in fluid communication with said top groove portion, and a liquid outlet that is in fluid communication with said bottom groove portion, and
a liquid-retaining mechanism that includes a liquid-retaining net clinging to said groove-defining surface so that liquid introduced into said top groove portion via said liquid inlet forms a liquid membrane on said liquid-retaining net through capillary action;
wherein gas introduced into said lower guiding groove via said gas inlet flows into said upper guiding groove through said passage groove with airborne components contained therein dissolving in the liquid membrane; and
wherein the liquid containing the dissolved airborne components flows into said bottom groove portion and exits said sampling device via said liquid outlet.

2. The sampling device of claim 1, wherein said liquid-retaining mechanism further includes an anchor plate mounted to said second plate, and disposed adjacent to said top groove portion for holding said liquid-retaining net on said passage groove to enable said liquid-retaining net to cling on said groove-defining surface.

3. The sampling device of claim 1, wherein said liquid-retaining net has 100 to 400 meshes per square inch.

4. The sampling device of claim 1, wherein each of said upper and lower guiding grooves is defined by a horizontal groove surface perpendicular to said first abutment surface, and an inclined groove surface extending from said horizontal groove surface to said first abutment surface, each of said gas outlet and inlet extending through said horizontal groove surface that defines the respective one of said upper and lower guiding grooves.

5. The sampling device of claim 1, wherein said groove-defining surface has an inclined bottom section bordering a bottom end of said bottom groove portion, said inclined bottom section having an end that is proximate to said side surface of said second plate and that is not higher than said liquid outlet, and an opposite end that is distal from said side surface of said second plate and that is above said liquid outlet so as to enable the liquid containing the dissolved airborne components to automatically flow to said liquid outlet.

6. The sampling device of claim 1, wherein said liquid guiding unit further includes a stop plate connected watertightly to said second plate and cooperating with said second plate to define said bottom groove portion.
